(19) United States
(12) Patent Application Publication
(10) Pub. No.: US 2004/0117871 A1
(43) Pub. Date: Jun. 17, 2004

(54) Plant growth regulation

(76) Inventors: Peter Meyer, Leeds (GB); Elena Zubko, Leeds (GB)

Correspondence Address: MYERS BIGEL SIBLEY & SAJOVEC, PO BOX 37428, RALEIGH, NC, 27627, US

(21) Appl. No.: 10/432,534

(22) PCT Filed: Nov. 26, 2001
(86) PCT No.: PCT/GB01/05175

(30) Foreign Application Priority Data
Nov. 25, 2000 (GB) 0028827.4
Jan. 13, 2001 (GB) 0100971.1
Oct. 5, 2001 (GB) 0123970.6

(51) Int. Cl. A01H 1/00; C12N 15/82; C07H 21/04

(52) U.S. Cl. 800/278; 536/23.6

(57) ABSTRACT

The present invention provides an endogenous nucleic acid encoding a cytokinin biosynthesis enzyme, products thereof and uses therefore and control thereof in the production of plants and/or plant cells that are capable of exhibiting a variety of advantageous characteristics associated with CK regulated processes.

The present invention relates to an isolated gene, uses thereof and a method of genetic manipulation, means therefore and products thereof especially for use in plant growth, senescence regulation and in controlling plant responses to environmental conditions.

### BACKGROUND TO THE INVENTION

The biosynthetic origin of CKs (cytokinins) in plants has been the subject of intense investigation ever since the recognition of CKs as plant hormones. CKs have been associated with many biochemical/physiological events such as the regulation of cell division, cell cycles, cell activity, stimulation of nutrient mobilisation, delay of senescence, lateral branching, morphogenesis and light initiated maturation of chloroplasts. CKs are the plant hormones responsible for cell growth and especially for shoot development For example, it is known that plants with high auxin and low CK levels tend to over develop root systems, whereas plants with low auxin and high CK levels tend to over develop shoot systems. Thus, in a normal healthy plant the role of CK is complemented by other plant hormones.

Although several biosynthetic pathways for CKs have been suggested, none of the data available to date are conclusive. The enzymes involved in CK biosynthesis have not yet been purified and characterised in detail, nor have any plant genes encoding CK biosynthesis enzymes been identified. Indeed, the only evidence so far in plants is that a gene termed CKII, a histidine kinase homologue, has been implicated in cytokinin transduction, possibly as a result of encoding a free form CK receptor (1).

It is known that CKs occur both in a free form and bound to tRNAs. The known free forms are trans-zeatin, N⁶-(Δ²-isopentenyl)adenosine (i⁶ Ado) (2iP) and dihydrozeatin Evidence suggests that it is the free forms that are the effective form and that free CKs play the prominent role in CK activity. The biosynthetic contribution of bound tRNA-CK is believed to be relatively small, the free form CK being released only as a result of tRNA degradation. It is known from the prior art in non-plant material that synthesis of tRNA-CKs depends on at least one enzyme, that being tRNA[9R]iP37-synthetase (EC 2.5.1.8). This enzyme has been identified in E.coli, where it is encoded by two genes, specifically the miaA gene and the mod5 gene. Homologues have also been found in Agrobacterium. However, despite intensive research no genes encoding CK biosythesis enzymes have so far been isolated/identified in plants.

We have identified a gene, the activity of which is associated with plant shoot production and which we conveniently refer to as the “shooting gene”. Moreover, we have found surprising that the gene encodes a CK biosynthesis enzyme which produces different forms of CKs in plants and that manipulation of the gene and its expression products allows control of cellular processes such as plant growth and regulation of senescence.

In an entirely different field of technology, it is known in the cosmetics industry to use topical retinoids as skin protection agents and especially for the reduction of wrinkles and other effects of skin ageing. However, retinoids can cause burning, redness and peeling of the skin if applied in high doses or too often. Kinerase® uses an alternative active component, a plant cytokinin. An alternative plant extract that could be used to reduce the signs of skin ageing would be of immediate benefit to the industry and consumer alike.

### STATEMENT OF THE INVENTION

In its broadest aspect the present invention provides a plant gene encoding a cytokinin biosynthesis enzyme.

The present invention resides in the isolation and characterisation of a naturally occurring plant gene that encodes a plant enzyme whose activity is sufficient to produce active CKs in plants. We have found that the gene of the present invention predominantly encodes 2iP derivatives as opposed to zeatin and dihydrozeatin derivatives. Zeatin and dihydrozeatin are the enzymes encoded by a homologous gene in Agrobacterium, so that the observations we have made in plants are most unexpected.

According to a first aspect of the invention there is provided a nucleic acid molecule comprising the sequence set forth in SEQ ID NO:1 or a part thereof or a homologue thereof, which encodes a cytokinin biosynthesis enzyme.

Preferably, the cytokinin biosynthesis enzyme produces 2iP derivatives.

Preferably, the nucleic acid sequence hybridises under high stringency conditions to SEQ ID NO:1.

Preferably the nucleic acid molecule is isolated.

Preferably, the nucleic acid is of plant origin and may be derived from either monocotyledonous or dicotyledonous plants.

According to a further aspect of the invention there is provided a nucleic acid molecule comprising the sequence set forth in SEQ ID NO:7 or a part or variant thereof or a homologue thereof, which acts as a promoter for a nucleic acid encoding a cytokinin biosynthesis enzyme.

According to a yet further aspect of the invention there is provided a polypeptide or protein in SEQ ID NO:2 or functionally equivalent part or homologue or derivative thereof. The polypeptide of SEQ ID NO:2 has the sequence of a putative tRNA-IPT enzyme.

Preferably the polypeptide or protein is encoded by a nucleic acid molecule of the invention.

The present invention therefore provides a nucleic acid encoding a cytokinin biosynthesis enzyme, the nucleic acid may be selected from the group consisting of:

(a) DNA having the nucleotide sequence given herein as SEQ ID NO:1 (which encodes the protein having the amino acid sequence given herein as SEQ ID NO:2), and which encode a cytokinin biosynthesis enzyme;

(b) nucleic acids which hybridise to DNA of (a) above (e.g., under stringent conditions) and which encode a cytokinin biosynthesis enzyme; and

(c) nucleic acids which differ from the DNA of (a) or (b) above due to the degeneracy of the genetic code, and which encode a cytokinin biosynthesis enzyme encoded by a DNA of (a) or (b) above.

of the present invention include those coding for proteins homologous to, and having essentially the same biological properties as, the proteins disclosed herein, and particularly the DNA disclosed herein as SEQ ID NO:1 and encoding the protein given herein SEQ ID NO:2. This definition is intended to encompass natural allelic variations therein. Thus, isolated DNA or cloned genes of the present invention can be of any plant species of origin. Thus, DNAs which hybridise to DNA disclosed herein as SEQ ID NO:1 (or fragments or derivatives thereof which serve as hybridisation probes as discussed below) and which code on expression for a protein associated with cytokinin biosynthesis (e.g., a protein according to SEQ ID NO:2) are included in the present invention.

Conditions which will permit other DNAs which code on expression for a t-RNA CK polypeptide or protein to hybridise to the DNA of SEQ ID NO:1 disclosed herein can be determined in accordance with known techniques. For example, hybridisation of such sequences may be carried out under conditions of reduced stringency, medium stringency or even stringent conditions (e.g., conditions represented by a wash stringency of 35-40% Formamide with 5×Denhardt's solution, 0.5% SDS and 1×SSPE at 37° C.; conditions represented by a wash stringency of 40-45% Formamide with 5×Denhardt's solution, 0.5% SDS, and 1×SSPE at 42° C.; and conditions represented by a wash stringency of 50% Formamide with 5×Denhardt's solution, 0.5% SDS and 1×SSPE at 42° C., respectively) to DNA of SEQ ID NO:1 disclosed herein in a standard hybridisation assay. See, e.g., J. Sambrook et al., Molecular Cloning, A Laboratory Manual (2d Ed. 1989) (Cold Spring Harbor Laboratory). In general, sequences which code for proteins of the present invention and which hybridise to the DNA of SEQ ID NO:1 disclosed herein will be preferably at least 75% homologous, 85% homologous, and even 95% homologous or more with SEQ ID NO:1. Further, DNAs which code for proteins of the present invention, or DNAs which hybridise to that of SEQ ID NO:1, but which differ in codon sequence from SEQ ID NO:1 due to the degeneracy of the genetic code, are also part of this invention. The degeneracy of the genetic code, which allows different nucleic acid sequences to code for the same protein or peptide, is well known in the literature. See, e.g., U.S. Pat. No. 4,757,006 to Toole et al. at Col. 2, Table 1.

Sequence identity: the similarity between two nucleic acid sequences, or two amino acid sequences, is expressed in terms of the similarity between the sequences, otherwise referred to as a sequence identity. Sequence identity is frequently measured in terms of percentage identity (or similarity or homology); the higher the percentage, the more similar the two sequences are. Homologues or orthologues of the protein, and the corresponding cDNA or gene sequence, will possess a relatively high degree of sequence identity when aligned using standard methods. This homology will be more significant when the orthologous proteins or genes or cDNAs are derived from species that are more closely related (e.g., human and chimpanzee sequences), compared to species more distantly related (e.g. human and C. elegans sequences).

Methods of alignment of sequences for comparison are well known in the art. Various programs and alignment algorithms are described in: Smith & Waterman Adv. Appl. Math. 2: 482, 1981; Needleman & Wunsch J. Mol.Biol. 48: 443, 1970; Pearson & Lipman Proc.Natl. Acad. Sci. USA 85: 2444, 1988; Higgins & Sharp Gene, 73: 237-244, 1988; Higgins & Sharp CABIOS 5: 151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio.24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis blastp, blastn, blastx, tblastn and tblastx. By way of example, for comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment may for example be performed using the Blast 2 sequences function, employing the PAM30 matrix set to default parameters (open gap 9, extension gap 1 penalties).

According to a yet further aspect of the invention there is provided use of a nucleic acid of the invention and/or protein or polypeptide encoded thereby in regulation of any one or more of the following processes; plant growth, shoot development, cell division, cell cycles, cell activity, stimulation of nutrient mobilisation, delay of senescence both in vivo and post harvesting, lateral branching, morphogenesis, increased pathogen tolerance and/or light initiated maturation of chloroplasts.

According to a yet further aspect of the invention there is provided a method of regulating any one or more of the following processes; plant growth, shoot development, cell division, cell cycles, cell activity, stimulation of nutrient mobilisation, delay of senescence, lateral branching, morphogenesis, increased pathogen tolerance and/or light initiated maturation of chloroplasts comprising genetically engineering a plant cell or tissue or plant or seed so as to incorporate a nucleic acid of the present invention into the genome.

Alternatively, the nucleic acid molecule of the present invention may be activated by, for example, an enhancer.

It will be appreciated that the nucleic acid of the present invention encodes a tRNA-IPT like enzyme and that manipulation of the nucleic acid and/or any product thereof will allow any one or more of the plant biochemical processes in which CKs play a role to be regulated/manipulated.

A further advantage of the present invention is that the nucleic acid is a naturally occurring endogenous molecule or a variant thereof.

According to a yet further aspect of the invention there is provided use of the nucleic acid and/or polypeptide encoded thereby in the activation of a target gene or genes.

It will be appreciated that CKs may act to trigger other plant processes by activation at the molecular level. The present invention therefore provides use of a putative tRNA-IPT enzyme in activation of other plant genes.

According to a yet further aspect of the invention there is provided use of the nucleic acid of the present invention and/or protein or polypeptide encoded thereby in regulating flood tolerance in plants.

Flooding is one of the most serious environmental stresses that affect plant growth and productivity. Flooding causes premature senescence which results in leaf chlorosis, necrosis, defoliation, cessation of growth and reduction of yield. Cytokinins can regulate senescence and by up-regulation of the levels of cytokinin the present invention is of particular advantage to plant growers in areas prone to floods.

According to a further aspect of the invention there is provided a vector which has inserted therein a nucleic acid molecule of the present invention.

Preferably, the vector is bacterial or viral in origin.

Preferably, the vector is an Agrobacterium transformation vector, however it will be appreciated that other vectors may also be used. Plant cells whose genome has been modified using a vector of the invention in practice comprise a fragment of such a vector which comprises a residue of a nucleic acid molecule of the invention; typically contain a nucleic acid sequence of the invention flanked by sequences derived from the unmodified vector, i.e. sequences foreign to both the plant cells and the nucleic acid sequence of the invention.

Preferably, the vector also includes an enhancer element to stimulate transcriptional activity. An example of such an enhancer element is by way of example only a 20 bp transcriptional sequence position −90 to −427 from the RB (right border sequence) of a 35S RNA promoter of cauliflower mosaic virus. In some embodiments of the invention, a plant genome is modified by insertion of an enhancer to stimulate transcription of an endogenous cytokin biosynthesis enzyme coding sequence; transgenes containing a foreign enhancer operatively associated with an endogenous cytokin biosynthesis enzyme coding sequence are thus included in the invention, as well as plant cells, plant tissues, seeds and plants containing such transgene sequences.

Preferably the enhancer is capable of stimulating transcriptional activity by at least a 10 fold over basal level transcriptional activity. More preferably the enhancer stimulates transcription 10-100 fold over basal level transcriptional activity. More preferably still the enhancer stimulates transcription greater than 100 fold over basal level transcriptional activity.

According to a yet further aspect of the invention there is provided use of a vector having inserted therein a nucleic acid molecule of the present invention in identifying a marker gene or promoter.

Relatively few plant promoters have been identified and even fewer differentially expressed promoters are known. The coding region of the “shooting” gene of the present invention can be used to tag plant promoters, as integration of the shooting gene coding region at random positions of the genome would only cause expression and a selectable phenotype if the coding region were integrated near an endogenous promoter. Additionally, because the shooting gene effect can be observed locally, the gene of the present invention can also be used to tag promoters that are only active in certain tissues.

The vector of the present invention may also be used to select a specific or clean line where a marker gene has been deleted or removed or disabled.

Thus, preferably in one embodiment of the invention the nucleic acid molecule of the present invention is fused to a reporter gene domain. Advantageously, this allows selection of events where endogenous promoters have been tagged and thus express the shooting gene, to be analysed for the expression profile of the endogenous promoter using the reported domain.

Preferably, the vector is an expression vector conventionally adapted for eukaryotic gene expression.

Typically, the adaptation includes by way of example only, a transcriptional control sequence (promoter sequence) which mediates cell/tissue specific expression. These promoter sequences may be cell/tissue specific inducible or constitutive. An example of such a promoter is a 35S RNA promoter of cauliflower mosaic virus.

Controlled expression of a gene producing CK effects is of special importance for the manipulation of crop plants. For example, expression of the gene products including polypeptide(s) and/or protein(s) encoded by the nucleic acid of present invention can cause efficient shoot development from callus, leaves or other tissue, without the need for exogenous growth hormones. Thus, in one embodiment of the invention the expression of gene products can be induced so as to overcome the poor shooting potential of certain species (recalcitrant plants) that has limited the success and speed of transgene technology for certain those species. Moreover, multiple shoot induction can be induced for crops where it is economically desirable to produce as many shoots as possible.

We have shown that the shooting/growth stimulating effect of the gene/gene products is/are quantity dependent. Accordingly, variants of the transformed line with reduced expression of the gene also show a reduced phenotype. In another embodiment of the invention, this effect can be used so as to elicit the effects of the gene only during certain developmental stages, and to switch the effect off in other stages where the gene-specific effect is not or is no longer desirable. Control (i.e. switching between on/off states) of the gene's expression can be obtained via use of inducible or tissue-specific promoters. Alternatively, the gene could be inverted or deleted using site-specific recombinases, transposons or recombination systems, which would also turn off gene-specific effects.

According to a yet further aspect of the invention there is provided a plant cell and/or plant tissue and/or a plant and/or a plant seed containing a transcriptionally activated/activatable form of the nucleotide molecule of the present invention, and optionally further including any of the preferred features as hereinbefore described.

According to a yet further aspect of the invention there is provided a plant cell and/or plant tissue and/or plant cell and/or plant seed comprising a recombinant transgene integrated into its genome characterised in that the transgene comprises the nucleic molecule of the invention. Such genes themselves form an aspect of the invention.

According to a yet further aspect of the invention there is provided a plant cell and/or plant tissue and/or plant and/or plant seed having integrated or inserted into its genome at least one more copy of a sequence encoding a nucleic acid molecule of the present invention than its naturally occurring counterpart. It is envisaged that the number can be in the range of 1-10 additional copies. There may be for example 1, 2, 3, 4, 5, 6, 7, 8, 9 or 10 or more copies of the encoding sequence.

Preferably, the plant cell and/or plant tissue and/or plant and/or plant seed has multiple copies of the sequence encoding the nucleic acid of the present invention.

A plant generated from a plant cell and/or plant tissue and/or plant seed which contains a transcriptionally activated/activatable sequence encoding a nucleic acid molecule of the present invention or a vector containing a nucleic acid molecule of the present invention.

A plant generated from a plant cell and/or plant tissue and/or plant seed which contains at least one additional copy of a sequence encoding a nucleic acid molecule of the present invention or a vector containing a nucleic acid molecule of the present invention.

Preferably the plant is a crop and more preferably is a cereal crop or grass.

The invention includes a plant material selected from a plant cell and/or plant tissue and/or plant and/or plant seed whose genome contains recombinant DNA comprising the sequence of a nucleic acid of the invention. It will be appreciated in this respect that in the instance of inserting the nucleic acid of the invention using a vector, for example bacterial vector such as e.g. an Agrobacterium vector, the nucleic acid is typically flanked by foreign (vector) nucleic acid sequences and that its presence in a plant cell can be determined by the presence of such foreign genetic material. In other instances, the nucleic acid of the invention is operatively associated with an enhancer.

According to a yet further aspect of the invention there is provided use of a vector or recombinant transgene or enhanced expression of the nucleic acid of the present invention in the production of plants with any one or more of the following characteristics:

(i) reduced apical dominance;

(ii) hormone-independent regeneration of shoot from calli and leaves;

(iii) reduced height (internode length);

(iv) increased leaf/stem ratios

(v) delayed flowering

(vi) delayed senescence

(vii) increased insect tolerance

(viii) increased flooding tolerance

(ix) increased life span of the plant

(x) increased vase and shelf life of plant parts and;

(xi) increased pathogen tolerance.

A method of producing a genetically modified plant with any one or more of the characteristics hereinbefore described comprising inserting a nucleic acid of the present invention or a vector or a recombinant transgene containing it into its genome or causing the nucleic acid expression to be enhanced above basal levels.

It will be appreciated that the benefit of a characteristic such as reduced apical dominance can result in the production of multiple shoots from one explant, generation of bushy phenotypes with reduced sensitivity to wind and/or rain and the production of plants with increased flower number to increase fruit/seed yield.

A plant having the characteristic of hormone-independent regeneration of shoot from calli and leaves will allow improved regeneration of plants from calli, production of somatic clones from leaves for micro-propagation and mass production, use of a tagged gene as a new selectable marker, use of the coding region of a tagged gene for identifying a promoter after random integration into plant genomes.

The characteristic of reduced height (internode length) and increased leaf/stem ratios provides the benefit of producing shorter plants with increased resistance to wind/rain, production of plants with increased leaf/stem ratios for animal feed especially useful for grasses and production of plants or cell lines with improved potential for expression and storage of recombinant enzymes in leaf tissue.

The characteristic of delayed senescence would advantageously improve vase and shelf life of flowers and crops in addition to extending the lifetime of cut flowers and the period of harvest to display of crop products.

The present invention provides an endogenous nucleic acid encoding a putative tRNA-IPT enzyme, products thereof and uses therefore and control thereof in the production of plants or plant cells that are capable of exhibiting a variety of advantageous characteristics.

According to a yet further aspect of the invention there is provided use of the nucleic acid of the present invention as a probe for selection of plants that exhibit enhanced levels of CK biosynthesis enzymes.

Preferably, the plants are naturally occurring.

It is envisaged that the nucleic acid of the invention will be particularly useful in identifying plant varieties that have high endogenous CK enzymes so that a plant variety may be selected for specific climatic and/or environmental conditions. For example, a particular variety of potatoes may be selected for a season or area where it is desired to have a rapid crop production. Alternatively, a particular flower may be selected where it is desired to maintain or produce early/late bloom.

According to a yet further aspect of the invention there is provided use as a cosmetic the nucleic acid as hereinbefore described and/or its expression products and/or a plant or plant material and/or plant extract which includes a transcriptionally activated/activatable form of the nucleic acid molecule or whose genome contains recombinant DNA comprising the sequence of a the nucleic acid. In addition the invention provides a cosmetic preparation comprising the nucleic acid as hereinbefore described and/or its expression products and/or a plant or plant material and/or plant extract which includes a transcriptionally activated/activatable form of the nucleic acid molecule or whose genome contains recombinant DNA comprising the sequence of a the nucleic acid and further including a suitable carrier, excipient, diluent or base or foundation product.

For example the cosmetic preparation may preferably further comprise molecules selected from the group consisting of elastin, elastin fragments, elastin-glycolic acid, collagen, collagen fragments, yeast extracts, skin respiratory factor, glucosamine, glucosamine sulfate, hyaluronic acid, hyaluronate, chondroitin sulfate, cholic acid, deoxycholic acid, ginseng extract, aloe vera powder, aloe vera oil, RNA and DNA fragments, ascorbyl palmitate, ascorbic acid, retinal palmitate, 7-dehydroxy cholesterol, vitamin E tocopherol, vitamin E lineolate, panthenyl ethyl ester, glycerol ceramides, glycogen, DL-pyroglutamic acid, urea, sodium lactate, lactate, glycerin, sorbitol, oils of borage, evening primrose, black currant, almond and canola, vanishing cream, cholesterol, flavenoids, witch hazel, chamomile, parsley, hibiscus, capric and caprylic triglycerides, amino acids, allantoin, sodium, calcium, potassium, phosphate, chloride, sodium lactate, alpha hydroxy acids, cocoa butter, coconut oil, jojoba oil, safflower oil, wheat germ oil, sesame oil, selachyl alcohol, shark oil, cerebrosides, proanthocyanidin, farnestol, candelellila, carnuba wax, vitamin E nicotinate, manganese ascorbate, zinc, oleyl alcohol, polysorbate 80, spermaceti, glycerol monostearate, beeswax, silicone oil, paraffin wax, ozokerit E, and PEG 75 lanolin.

Preferably, the cosmetic preparation is in the form of a lotion, salve, cream, liposome, spray, micelle or gel.

According to a yet further aspect of the invention there is provided a method of improving skin/hair/fingernail/toenail condition moisturising skin/hair/fingernail/toenail and/or preventing wrinkles in keratinous structures and/or other ageing effects of the skin comprising applying topically a cosmetic preparation as hereinbefore described.

### BRIEF DESCRIPTION OF THE FIGURES

FIG. 1 represents a schematic representation of the enhancer method.

FIG. 2 illustrates Petunia transformants (Ph-sh) grown on hormone-free medium and appropriate controls.

FIG. 3 illustrates examples from a line Ph-sh.

FIG. 4 illustrates the expression analysis of the tRNA-IPT homologue gene.

FIG. 5 illustrates the induction of shoot development into tobacco of the re-isolated T-DNA with its integration region.

FIG. 6 illustrates improved vitality and tolerance against infection.

FIG. 7 illustrates transfer of the shooting-gene into leaf disks of Nicotiana tabacum (A), Atropa belladonna (B), Petunia hybrida (C) and Solanum tuberosum (D).

FIG. 8 illustrates a wild type plant of similar age (left) grown in parallel with line Ph-sh2 (right). The plants are shown after 53 days (A), 70 days (B), 84 days (C) and 129 days (D). Compared to wild type, Ph-sh2 shows a more bushy phenotype with reduced apical dominance. Flowering and senescence is delayed and the plant is less susceptible to pathogen infections. The line Ph-sh2 is the same line shown in FIG. 6 of the present application application.

FIG. 9 illustrates plasmid rescue from Ph-sh.

FIG. 10 illustrates the Sho-gene encodes a protein with homology to putative tRNA-IPTases.

FIG. 11 shows expression analysis of the Sho-gene in wild-type plants and in transgenic lines Ph-sh and Ph-sh2.

FIG. 12 shows expression of the Sho-gene in Nicotiana tabacum transformants. Northern blot analysis showed that enhanced shoot production in the transformants correlated with expression of the Sho-transgene.

FIG. 13 shows phenotypes of transgenic tobacco plants generated after transformation with Sho-constructs.

FIG. 14 illustrates improved shelf life in harvested material.

FIG. 15 illustrates improved vase life of stem cuttings.

FIG. 16 illustrates reduced pathogen infection from stem cuttings.

### DETAILED DESCRIPTION OF THE INVENTION

### Materials and Methods

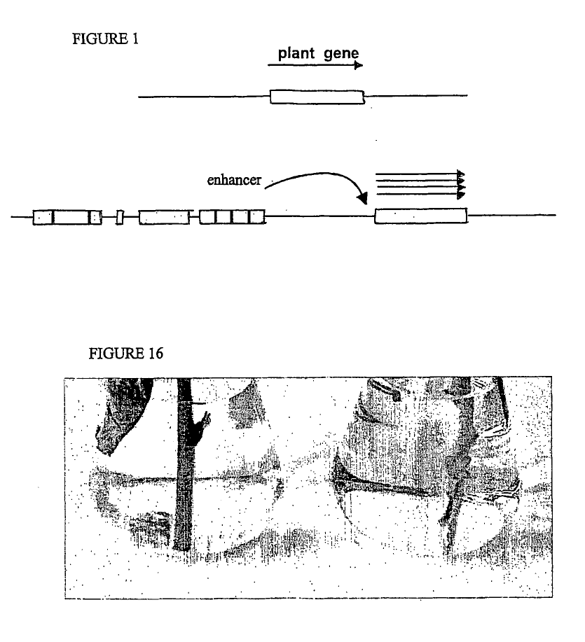

FIG. 1; illustrates the method employed in the present invention. An enhancer (1), located on a T-DNA, is placed into random positions of the genome. Once the enhancer is inserted next to a plant gene (2), the enhancer induces very strong expression of this adjacent gene, which may normally not be transcribed at all, at low levels or only in certain tissues. Transformants are screened for abnormal phenotypes that may reflect the strong activity of plant genes located adjacent to the enhancer. This close proximity allowed cloning of the gene of the present invention.

Agrobaclenrium Strain and Growth Conditions

All transformation experiments were performed using Agrobacterium tumefaciens strain GV 3101 (pMP90RK) carrying the T-DNA vector pPCVIC En4 Hpt (Fritze et al., 1995). A single colony of Agrobacterium from a 3-5 days old plate was incubated in 5 ml YEB medium. A liquid culture was grown at 29° C. for 24 h-36 h until the OD₆₀₀ reached 0.6-1.0. 1 ml of bacterial culture was collected by centrifugation for 1 min and resuspension in protoplast culture medium. Bacteria cells were then used to inoculate protoplast suspension.

Plant Material and Transformation

The cultivar Pink Wave of Petunia hybrida (purchased from Thompson and Morgan,UK) was grown aseptically from sterilised seeds and was propagated as shoot culture on MS medium. Plants were maintained at 22° C. in a 16-h photoperiod and were subcultured every two months. Protoplasts were isolated from 5-6 week old plant using an enzyme solution containing 0.4% Celulase “onozuka” R-10 0.4% Macerozyme R-10 (Yakult Honsha Co., LTD), 0.06M CaCl₂ and 0.375M mannitol. Protoplasts were plated in culture medium V-KM (Binding et al., 1984) at a density 10⁵ cells/ml. In 3-5 days, when protoplasts started to divide, 50 μl of Agrobacterium culture was added to the cell suspension. Protoplasts were co-cultivated with Agrobacterium for 3 days in the dark at 22° C. After 3 days protoplasts were washed with W₅ solution and were cultivated for 5-6 days in the same culture medium with claforan (250 mg/l) to prevent bacteria growth. Within 5-6 days the suspension of protoplast-derived microcolonies was diluted in medium C (Muller et al., 1983) with claforan (200 mg/l). Hygromycin (20 mg/l) was added at this stage to select transformants. Every 10 days colonies suspensions were diluted by fresh medium C supplied with antibiotics. After 30 days macrocolonies resistant to hygromycin were transferred on shoot induction medium containing MS basal salts, 20 g/l sucrose, 53 g/l mannitol, Wetmore's vitamins, 2 mg/l BAP and 0.9 mg/l IAA. Regenerants were transferred on MS medium and screened for phenotype.

DNA Preparation

Genomic DNA was isolated as described by Rik van Blokland et. al (1998).

T-DNA Rescue

Twenty μg of genomic DNA was digested with SacI. Digested DNA was purified by phenol/chloroform extraction and precipitated by ethanol. The DNA pellet was dissolved in water at a final concentration 20 μg/ml and autoligated. After ligation DNA was precipitated by iso-propanol, washed by 70% ethanol and dissolve in water at a concentration of 100 μg/ml. One μl of DNA was used for electroporation of E.coli Electro MAX DH10B cells. Transformants were selected on LB plates with ampicillin.

For sequencing analysis inserts were cut out from rescued plasmid with EcoRV and SacI and were re-cloned into BlueSKp.

Sequencing Analysis

Sequencing analysis was performed in six sequencing reactions starting with primers M13F, M13R, followed by sequencing reactions with four insert-specific primers:

(SEQ ID NO:3)

AF2-5′-ACA TGT CGT CAT CCA CTG TAG TAA-3′

(SEQ ID NO:4)

AF3-5′-AGG TTT TCG GAT CCC GGT TTG GAA C-3′

(SEQ ID NO:5)

AR2-5′-GTA TTA TAC AAT CCA AAG ATT GAG-3′

(SEQ ID NO:6)

AR3-5′-CAC CAA AAT GAA CTA GAG TGG GAT A-3′

Analysis of sequencing data is done using Blast Search program.

RNA Analysis

Total RNA was isolated from leaves of 6 weeks old plants as described by Logemann (1987). Twenty μg of total RNA was fractionated by electrophoresis in an 1% agarose denaturing formaldehyde gel. After electrophoresis the RNA was blotted on a GeneScreen transfer membrane, crosslinked by UV irradiation and hybridised with a ³²P-labelled probe at 65° C. according to Koes et al., (1987). The complete insert was used as probe.

Results

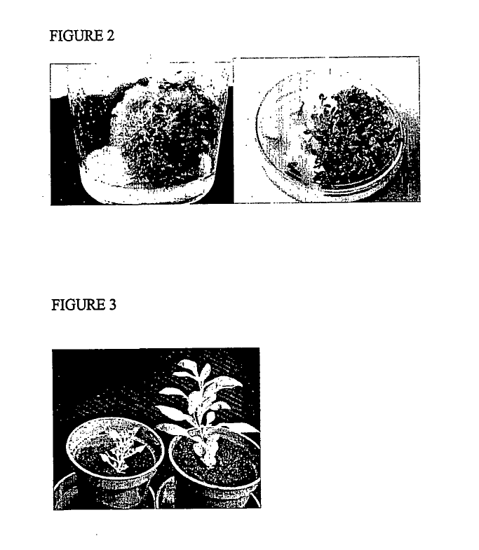

From among 5400 transformants, we selected one line (Ph-sh) because it was fast growing and continuously produced adventitious shoots on hormone-free medium. We have shown that when the line is transferred to new medium, leaves of these shoots again produce adventitious shoots but never morphologically normal plants (FIG. 2).

FIG. 2 (left-hand side example) illustrates a Petunia transformant (Ph-sh) which grows very fast and continuously produces adventitious shoots on hormone-free medium. The right hand example shows that when leaves of line Ph-sh are placed on hormone-free medium, they continue to produce multiple shoots (right hand side of dish) whereas control leaves don't survive (left-hand side of dish)

FIG. 3 illustrates examples from a line Ph-sh. We isolated a shoot that no longer showed the “shooting-lawn” phenotype as observed for Ph-sh but displayed a less severe shooting phenotype. This derivative, referred to as Ph-rev, is shown in the left hand pot, a wildtype control line is shown in the right hand pot, in order to demonstrate that Ph-rev has a phenotype intermediate between wildtype and Ph-sh.

Recloning of the T-DNA Integration Region

We postulated that if the shooting phenotype reflects the activity of an endogenous gene that is activated by the enhancer of the T-DNA, we would find an open-reading frame near the enhancer, encoding a mRNA that is significantly enhanced in the shooting mutant, compared to wildtype plants.

To confirm this, we recloned about 3 kb of the genomic region that was located next to the right border of the T-DNA. The strong enhancer is located directly at the right border. The region was isolated via plasmid rescue following digestion of genomic DNA of the shooting line, autoligation of the digested fragments and transformation into E.coli Electro MAX DH10B cells. Only religated circular fragments that comprised the T-DNA were able to propagate in E.coli, as only these fragments contained a bacterial origin of replication and an ampicillin resistance gene that was located on the T-DNA. The isolated plasmid therefore contains the T-DNA region with the origin of replication, the amp-resistance gene and the right border and a 3 kb region of genomic DNA directly adjacent to the right border.

A 3 kb insert was isolated from rescued plasmid DNA and re-cloned into BlueSKp. Sequencing showed that the region contains an ORF 1, located between 253-1305 bp from the right border (SEQ ID NO:1), which encodes a 350aa protein (SEQ ID NO:2) with strong homology to a putative tRNA-isopentenyl transferase isolated from Arabidopsis thaliana. The protein sequence of SEQ ID NO:2 shows highest homology to:

1. tRNA-arab putative tRNA isopentenyl transferase; 11395-10322 [Arabidopsis thaliana] ACCESSION AAF16599

2. tRNAarab2 tRNA isopentenyl transferase-like protein [Arabidopsis thaliana].ACCESSION BAB02956

3. tRNAarab3 hypothetical protein F22K18.150 [Arabidopsis thaliana].ACCESSION T05569

4. tRNAarab4 hypothetical protein; 86035-87063 [Arabidopsis thaliana].ACCESSION AAG12736

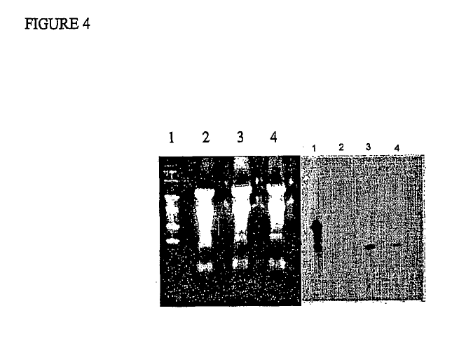

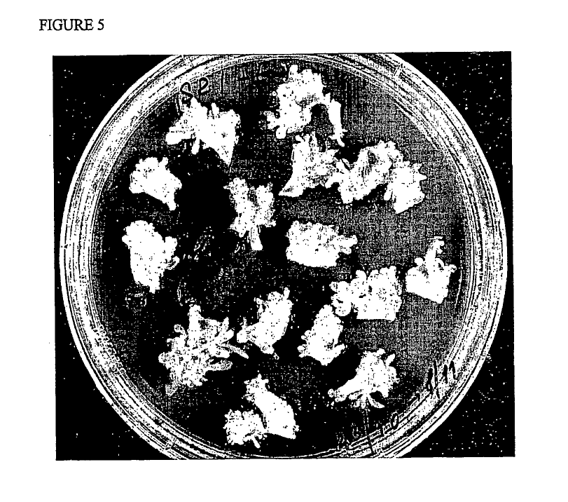

Expression of this putative tRNA-IPT homologue gene was compared in wildtype plants, in the Ph-sh line and in Ph-rev, a plant derived from Ph-sh that showed a less severe shooting phenotype (FIG. 3). FIG. 4 shows the expression analysis. No expression of the gene is detectable in wildtype but the Ph-sh line shows strong expression. This supports the assumption that the expression of the gene is enhanced due to the activity of the adjacent enhancer. In line Ph-rev, which shows a less severe shooting phenotype, expression of the gene is reduced (probably due to expression variation as it is frequently observed among plant transformants). This observation supports the assumption that the shooting phenotype is directly caused by the activity of the gene, and that the severity of the phenotype is quantity dependent.

FIG. 4 illustrates the expression analysis of the putative tRNA-IPT homologue gene in leaves of wildtype (2), Ph-sh (3) and Ph-rev (4). Size markers are shown in lane 1. Total RNA is shown on the left. Hybridisation signals to a putative tRNA-IPT homologue probe are shown on the right.

Phenotypic Effects of the Petunia Line Ph-sh and its Derivative Ph-rev

Ph-sh shows a stronger expression of the petunia gene adjacent to the integrated T-DNA enhancer compared to Ph-sh. Ph-sh still expresses this gene but at a lower level. Wildtype plants do not show any expression of the gene in leaves that could be detected in Northern blots.

Transfer of Re-isolated T-DNA with its Integration Region into Tobacco

The T-DNA from the petunia shooting line, together with a 2737 bp genomic petunia region located next to the right border of the T-DNA was inserted into an Agrobacterium transformation vector. This vector was used for leaf disk transformation. Leaves were put on MS medium without any hormones and on 250 mg/l claforan to inhibit growth of Agrobacterium. The leaves produced multiple shoots, indicative for a hormonal function of the construct, see FIG. 5. Untransformed leaf disks are normally unable to produce shoots on hormone-free medium, as hormones are essential for shooting. It is not clear if all shoots contain the transgene, as it is theoretically possible that the transgene is produced in certain transformed cells where it produces cytokinines, which can be exported to untransformed regions where they induce shooting.

Expression of the Shooting Gene

We have shown that the endogenous shooting gene is expressed at very low levels (about 200 fold lower that the level found in the shooting line that carries the four enhancers). The endogenous gene is most strongly expressed in roots. Compared to roots, expression levels are about 50% in leaves and 12% in plant tips. This defines the shooting gene promoter as a promoter with low activity, which can be a useful tool if expression of very low levels of particular transgenes is required. The sequence of this promoter (position 1-252 relative to the T-DNA right border) is set forth in SEQ ID NO:7.

Improved Vitality, Shelf and Vase Life and Tolerance Against Infection

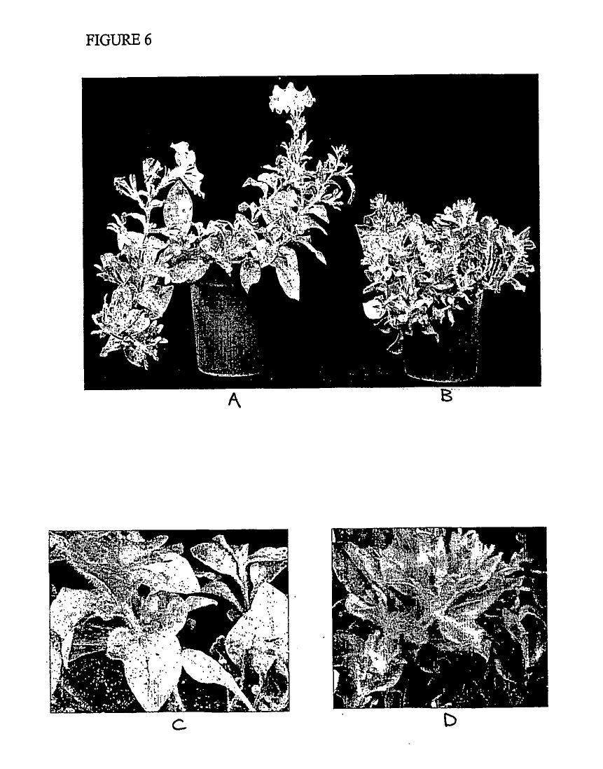

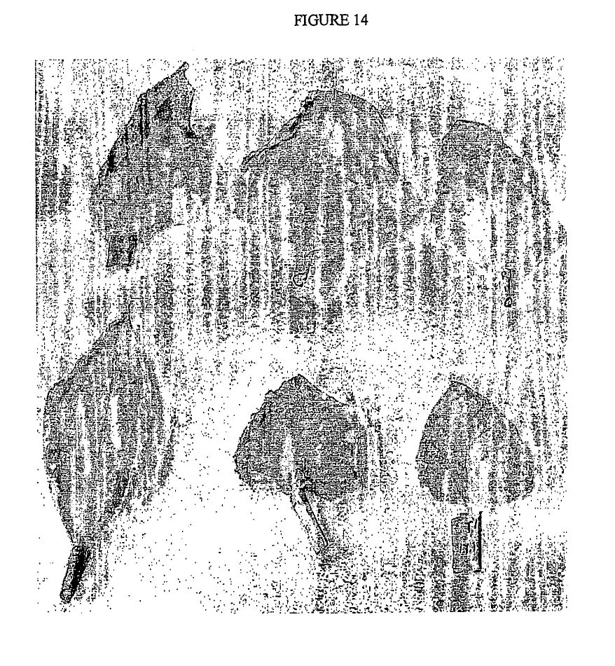

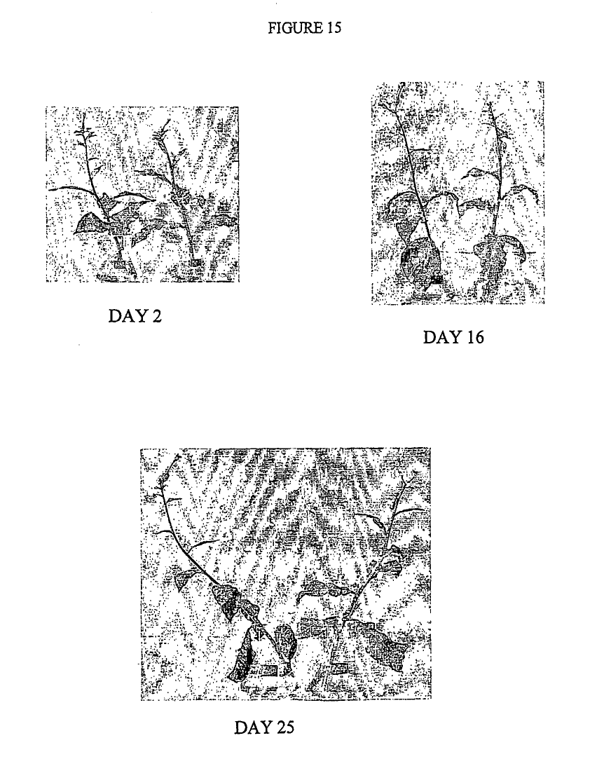

With reference to FIG. 6, we grew a petunia wildtype plant (left hand side termed Pink Wave) and the Ph-rev shooting line (the shooting line with the mild shooting phenotype) in the greenhouse under identical conditions. The wildtype, although of the same age, started to senesce much earlier than the Ph-rev. In addition, with reference to FIG. 16, we noticed that the wild type (left hand side stem cutting) was also more severely affected by pathogen infections after 25 days than the stem cutting on the right hand side (CK-tobacco). It therefore appears that the nucleic acid of the present invention may not only influence vitality and life span but in addition it may improve pathogen tolerance. With reference to FIG. 14 there is shown 16-day-old tobacco leaves from wildtype plants (top row) and plants with active shooting gene. Plants with the active shooting gene show improved shelf life of harvested material. With reference to FIG. 15 there is shown stem cuttings of wildtype tobacco plants (left hand side) and CK-tobacco cuttings (right hand side) at 2, 16 and 25 days post-harvest. The CK-tobacco cuttings at 25 days are green and healthy illustrating that the shooting gene improves shelf life.

Cytokinin Analysis

Cytokinin analysis has shown that the shooting line shows increased levels of isopentenyladenine-derivatives (Isopentenyladenosinephosphate, 3 fold enhanced; Isopentenyladenosine, 9 fold enhanced, Isopentenyladenine, 3 fold enhanced; Isopentenyladenine-N7-glucoside, 13 fold enhanced; Isopentenyladenine-N9-glucoside, 120 fold enhanced). Accordingly, the data shows that the shooting gene causes an increase in cytokinin levels the levels and isopentenyladenine-derivatives are discussed in greater detail hereinafter.

Transgenic Effects

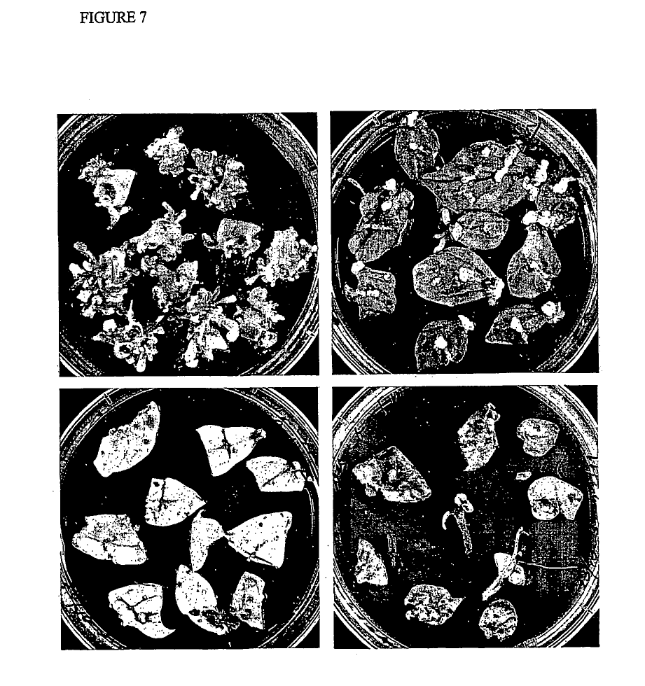

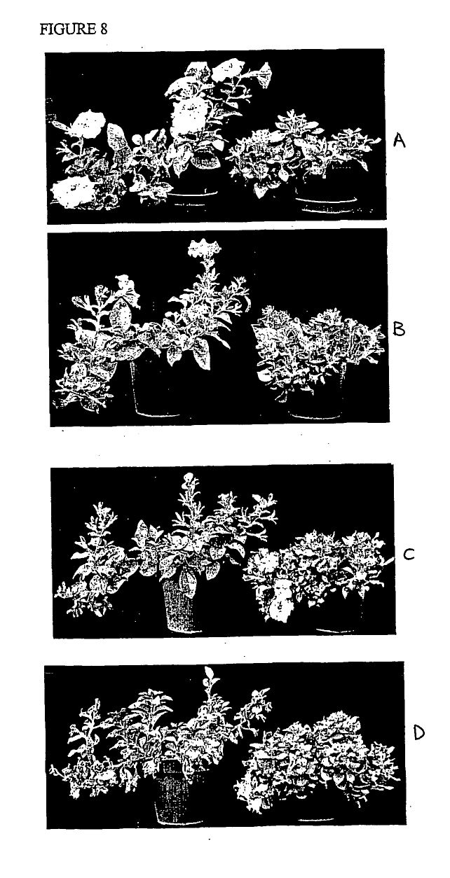

With reference to FIG. 7, the shooting-gene under the control of the 35S promoter was transferred into leaf disks of Nicotiana tabacum (A), Atropa belladonna (B), Petunia hybrida (C) and Solanum tuberosum (D). In all four species, shoots developed on hormone-free medium.

Transfer of the shooting gene, under the control of the 35S promoter, or under the control of its own promoter, linked to four enhancer copies of the 35S promoter, produces cytokinin effects in at least the following species:

Nicotiana tabacum

Petuniahybrida

Atropa belladonna

Solanum tuberosum

This evidence demonstrates that the shooting gene is responsible for the cytokinin effects, and that the effects can be transferred into other species.

We have found that the strength of the cytokinin effects strictly correlates with the transcript levels of the shooting gene. Transformation produces a variety of transformants that differ in expression levels of the transgene, due to position effect. Transformants with low expression levels also produce milder effects than transformants with higher levels. This demonstrates that the gene causes quantitative effects, and that modulation of expression levels allows a modification of the severity of cytokinin effects.

Isolation of the Sho-gene Responsible for the CK-specific Phenotype

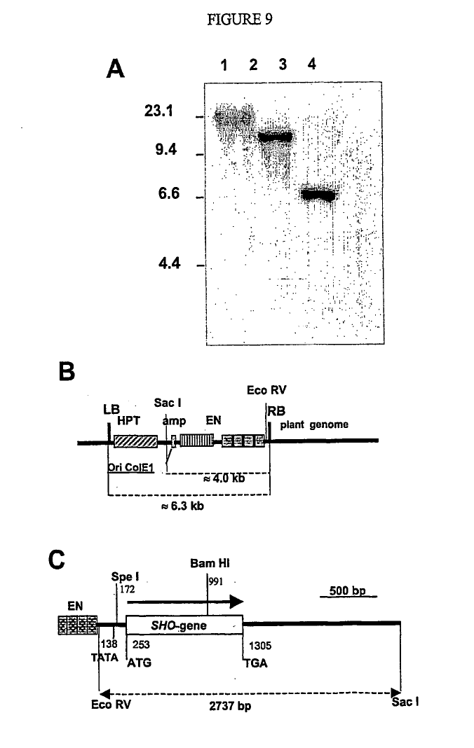

FIG. 9 shows plasmid rescue from Ph-sh. FIG. 9(A) showsouthern-blot analysis of Ph-sh that revealed a single site integration of the T-DNA. Genomic DNA was digested with XhoI (lane 1), EcoRI (lane 2) and SacI (lane 3), and was hybridised with a 35S-enhancer probe. Lane 4 is the wild type control DNA digested with EcoRI. The 6.7 kb SacI-fragment was used for plasmid rescue. FIG. 9(B) shows a schematic map of the T-DNA region. SacI cuts inside the T-DNA next to the ampicillin resistance gene, 4 kb upstream of the right border. The SacI-fragment contains about 2.7 kb of genomic DNA. FIG. 9(C) shows the genomic structure of the Sho-gene region.

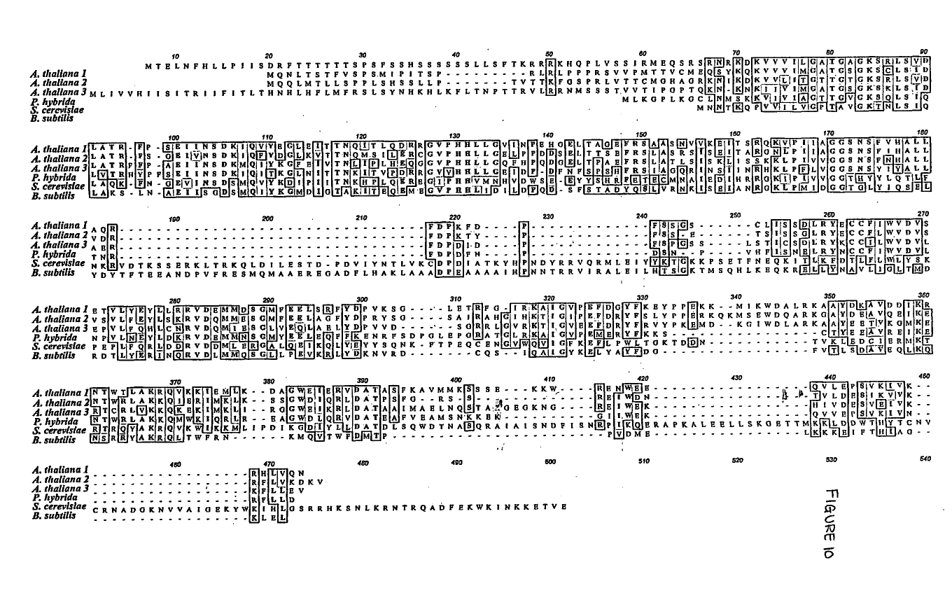

FIG. 10 shows the Sho-gene encodes a protein with homology to a putative tRNA-IPTases. Amino acid alignment (Thompson et al., 1994) of the Sho protein from P. hybrida with three tRNA-IPTases from A. thaliana (accession number AAF 16599, BAB02956 and AAG12736), with the Mod5p tRNA-IPTases from S. cerevisia (Accession number NP 014917) and with the MiaA tRNA IPP transferase from B. subtilis (Accession number O 31795). Identical residues are boxed in dark grey and conserved residues in light grey.

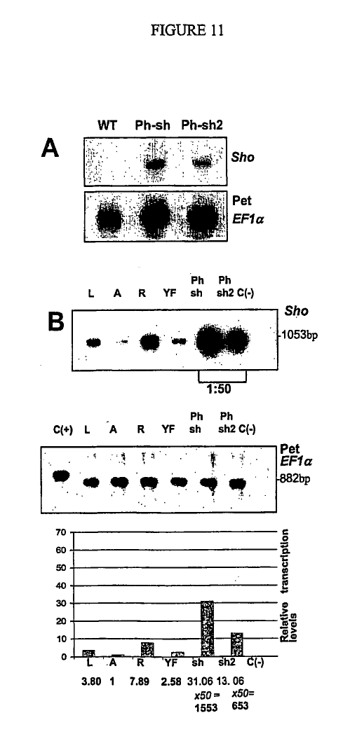

FIG. 11 shows expression analysis of the Sho-gene in wild-type plants and in transgenic lines Ph-sh and Ph-sh2. FIG. 11(A) shows total RNA that was isolated from leaves of 6-weeks old plants grown under aseptic conditions and tested for the presence of Sho-gene transcripts. Northern blot analysis fails to detect Sho-gene expression in a wild-type plant, while the gene is clearly expressed in lines Ph-sh and Ph-sh2. Expression is moderately lower in Ph-sh2, which is in accordance with the less severe phenotype of Ph-sh2. The Elongation factor 1α (Pet-EF1α) gene was used as a control for RNA loading. FIG. 11(B) shows RT-PCR analysis with specific primers for the Sho-gene and the Pet-EF1α-gene. Total RNA was isolated from leaves (L), apex (A), roots (R) and young flowers of 8 weeks old plants. RT-PCR products were separated and hybridised to Sho- and Pet-EF1α-specific probes. The lanes contain RT-PCR samples from wild-type leaves (L), apex (A), roots (R) and young flowers (YF), and from leaf tissue of Ph-sh and Ph-sh2. For the Sho-specific blot, RT-PCR samples of Ph-sh and Ph-Sh2 were diluted 1:50. For the Pet-EF1α-specific blot, all RT-PCR samples were loaded undiluted. C(−) indicates negative RT-PCR controls. C(+) indicates a Pet-EF1α genomic PCR-product as a positive control. All data were standardised on the corresponding Pet-EF1α value. A comparison of the relative transcription levels confirms the enhanced expression of the Sho-gene in Ph-sh and Ph-sh2. It also shows that, in wild-type plants, the Sho-gene is expressed at a low level in all tissues tested, and that wild-type expression levels are highest in roots.

Assuring that the CK-specific effects were quantitatively dependent on the expression of an endogenous gene, activated by the T-DNA enhancer tetramer, Ph-Sh2 could be an epigenetic variant of Ph-sh, with a reduced enhancer activity that would lead to a less severe CK-specific phenotype. We cloned the integration region near the right border of the T-DNA to search for open reading frames that could indicate the presence of an endogenous gene next to the T-DNA. Southern blot analysis (FIG. 9A) revealed that line Ph-sh contained one T-DNA insertion. SacI restriction produced a 6.7 kb fragment that contained the right border region, the origin of replication and the ampicillin resistance gene of the T-DNA, as well as 2.7 kb of the T-DNA integration region adjacent to the right border (FIG. 9B). The 6.7 kb SacI fragment was cloned after autoligation of the digested fragments and transformation into E.coli DH10B cells.

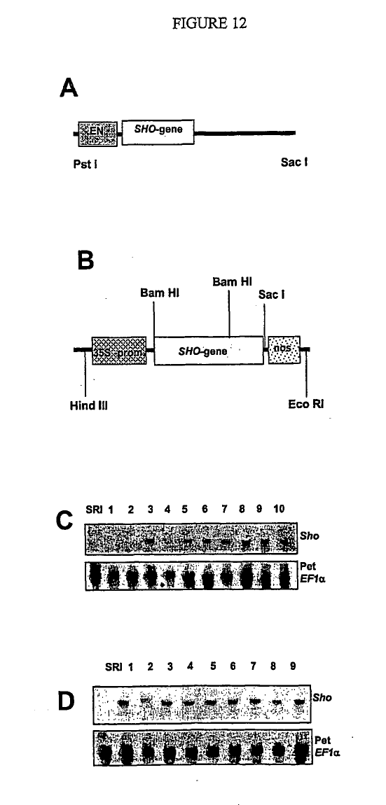

Sequence analysis showed that the region contained an ORF, located between 253 and 1305bp from the right border, which we labeled Sho-gene (FIG. 9C). The Sho-gene ORF encodes a 350aa protein with homology to the Mod5p tRNA-isopentenyl transferases from S. cerevisiae and the MiaA tRNA-isopentenylpyrophosphate transferases from B.subtilis (FIG. 10). It also shows strong homology to eight putative tRNA-isopentenyl transferases or tRNA-isopentenyl transferases-like proteins from Arabidopsis thaliana, three of which are shown in FIG. 10. Northern blot analysis did not detect significant levels of Sho-gene expression in wild type petunia, while the gene was clearly expressed in both transgenic lines, with slightly higher expression levels in line Ph-sh compared to its derivative Ph-sh2 (FIG. 12A). RT-PCR analysis showed that the Sho-gene is actually expressed at low levels in different tissues of wild type plants, with relatively highest expression levels in root tissue (FIG. 11B).

Expression of the Sho-gene Induces Hormone-free Shoot Development in Other Species

FIG. 12 shows expression of the Sho-gene in Nicotiana tabacum transformants. Northern blot analysis showed that enhanced shoot production in the transformants correlated with expression of the Sho-transgene. FIG. 12(A) shows vector A contains the complete rescued genomic fragment from Ph-sh and four 35S-enhancers elements (EN). FIG. 12(B) shows vector B, the coding sequence of the Sho-gene was inserted between the CaMV 35S promoter and the nos polyA region. FIG. 12(C) shows Northern blot analysis of vector A transformants. 1,2-transgenic plants that do not show a Sho-phenotype. 3-10-tansgenic plants with Sho-phenotypes of different intensity. FIG. 12(D) shows Northern blot analysis of vector B transformants. All 9 tested plants displayed a strong Sho-phenotype.

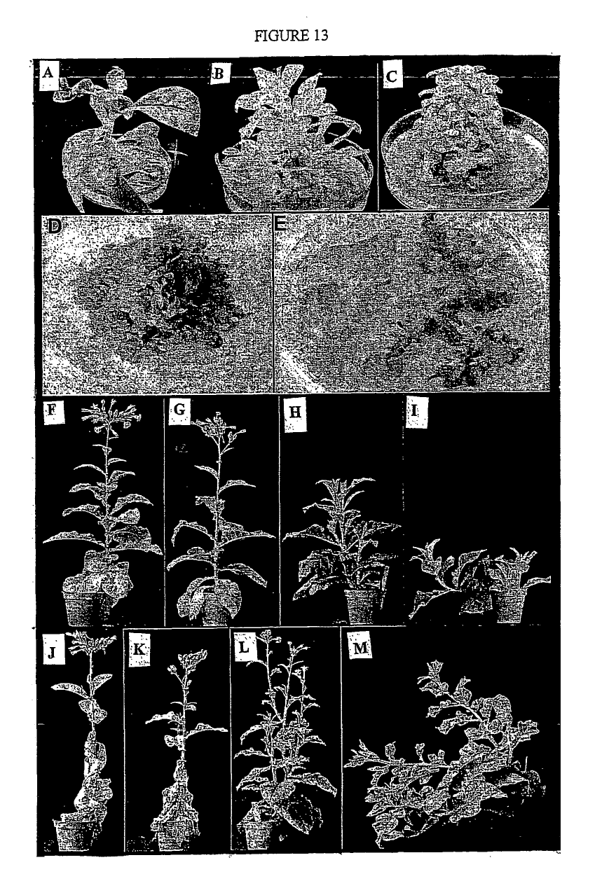

FIG. 13 illustrates phenotypes of transgenic tobacco plants generated after transformation with Sho-constructs.

In FIGS. 13(A-E) shows plants growing under sterile conditions. (A) Untransformed tobacco SRI, (B,C) Two construct A transformants with less (B) and more intensive phenotype (C). (D) Very strong shooting phenotype in a transgenic tobacco plant transformed with construct B. (E) Leaves of transgenic tobacco plants are able to produce shoots on hormone-free medium (right), in contrast to leaves of untransformed tobacco plants (left). (F-M) Plants grown in the greenhouse after 66 days (F-I) and 97 days (J-M). (F,J) Untransformed tobacco SRI. (G,K) Transgenic tobacco plant that does not express the Sho-gene. (H,L) and (I,M) two Sho expressing transformants that show reduced apical dominance, and delayed flowering and senescence.

To test if expression of the Sho-gene is sufficient to generate CK-specific effects, we designed two constructs (FIGS. 12A,B), which we transferred into Nicotiana tabacum. Selection of transformed tissue on MS hormone-free medium resulted in a very high number of tobacco regenerants but some of these lines displayed no obvious Sho-phenoytype and turned out to be none-transgenic. In contrast, all transformants regenerated on hormon-free medium with kanamycin were truly transgenic. Among 36 construct A transformants, 32 displayed Sho-phenotypes at different levels of intensity (FIGS. 13B,C). Three transformants with less intense phenotypes were able to root and to grow in soil (FIGS. 6H,I,L,M). Out of 15 construct B transformants, all had intense Sho-phenotypes (FIG. 6D). Northern blot analysis demonstrated a correlation between the appearance of Sho-phenotypes and expression of the Sho-gene (FIGS. 13C,D). Similar effects were observed when the Sho-gene was introduced into Petunia hybrida, Atropa belladonna and Solanum tuberosum (data not shown).

Sito-specific Phenotypes are Associated with Enhanced CK Levels

Plants of similar age, grown under identical sterile conditions, were assayed for 25 different CK compounds (Table 1). The results illustrated a high variability in CK composition depending on age and tissue type of the samples. If we account for this. variation and only concentrate on differences that are at least 10-fold, it becomes obvious that both Ph-sh lines have significantly enhanced levels of certain 2iP-type CKs, especially of the two glucosides 2iP7G and 2iP9G (Table 1A). Ph-sh tissue had higher levels of almost all 2iP-type CKs compared to Ph-sh2 tissue of the same age. In the tobacco transformants (Table 1B), we again observed a pronounced increase in 2iP7G levels. In contrast to petunia, however, 2iP9G concentrations were not increased.

In transgenic plants, the Sho-gene primarily enhances the accumulation of 2iP-type CKs, especially 2ip7G in petunia and tobacco, and, in addition, 2ip9G in petunia. This indicates that 2iP derivatives are the major products of the Sho-gene, and that these are efficiently channelled into the N-glucosylation pathway. This contrasts the effect of the ipt gene, which primarily enhances the levels of zeatin, zeatinribosides and zeatin riboside O-glucosides. The accumulation of individual CK-types is thought to depend on the pool of 2iP modifying enzymes and the differences in 2iP9G accumulation between petunia and tobacco illustrates that this pool can differ in different species. However, the different effects on CK-types in the same species (tobacco) transformed by the Sho-gene and the ipt gene argue in favour of two alternative routes of 2iP processing. This could indicate that Sho and IPT are active in different compartments of the cell, which have a different pool of modifying enzymes.

There also may be differences in the transport of Sho products and IPT products. From leaf disk transformations we isolated some shoots on hormon-free medium that turned out to be non-transgenic, when we did not include kanamycin selection. This suggests that Sho-gene activity can produce shoot-inducing substances that are able to migrate into adjacent tissue, while CK activity in ipt transformants may be more restricted to the site of synthesis. This observation we believe, makes the nucleic acid of the present invention particularly advantageous in the cosmetic industry since it is envisaged that plant tissue could be applied directly to skin and thus obviate the need to extract CK from plant tissue.

The identification and isolation of the Sho-gene of the present invention focuses the attention on the role of Sho-gene homologues in CK production, especially their regulation, and the mobility and location of the gene product. In wild type plants, we find very low but detectable levels of Sho-gene expression. In accordance with previous reports that roots are the main location for cytokinin production petunia root tissue contains the highest concentration of most CK-types (Table 1A) and the relatively highest expression levels of the Sho-gene are observed in root tissue. The gene is, however, also active in other tissues, especially in leaves, which have been shown to have a potential for CK synthesis.

TABLE 1A

| | 8 weeks old | | | | | | 10 weeks old | | | | | |
|---|---|---|---|---|---|---|---|---|---|---|---|---|
| | PW- | | | | | | | | | | | |
| | PW-wildtype | | Ph-sh | | Ph-sh2 | | Ph-wildtype | | | Ph-sh2 | | |
| | leaf | stem | leaf | stem | leaf | stem | leaf | stem | root | leaf | stem | root |
| 2iP | 2.2 | 1.0 | 8.0 | 0.6 | 1.2 | 1.4 | N | N | N | N | N | N |
| 2iPR | 2.0 | 4.4 | 22.4 | 20 | 2.0 | 19.6 | 0.6 | 0.4 | N | 1.4 | 12.2 | 3.6 |
| 2iPRP | 2.4 | 1.0 | 4.4 | 1.0 | 0.2 | 0.6 | 5.2 | 3.0 | 0.6 | 10.6 | 11.6 | 16.4 |
| 2iP7G | 6.0 | 11 | 105 | 39.8 | 45.8 | 23.0 | 7.8 | 20.0 | 20.0 | 101 | 77.8 | 144 |
| 2iP9G | 6.8 | 8.2 | 365 | 147 | 74.4 | 43.6 | 2.4 | 8.0 | 8.6 | 112 | 88.2 | 186 |
| Z | N | N | N | N | N | N | 0.2 | 1.6 | N | 2.4 | 1.4 | N |
| cZ | N | N | N | N | N | N | N | N | N | N | N | N |
| ZR | N | N | N | N | N | N | 2.0 | 0.4 | N | N | 0.6 | 0.4 |
| ZR | N | N | N | N | N | N | 3.0 | 0.6 | 0.8 | N | 0.6 | 0.4 |
| ZRP | N | N | N | N | N | N | 2.4 | 3.4 | 0.2 | 8.8 | 22.2 | 1.4 |
| cZRP | N | N | N | N | N | N | 3.4 | 2.8 | N | 4.8 | N | 0.4 |
| ZOG | N | N | N | N | N | N | 1.6 | 0.8 | 2.6 | N | 0.6 | 3.2 |
| cZOG | N | N | N | N | N | N | 4.2 | 3.0 | 12.2 | 9.8 | 6.8 | 15.2 |
| ZROG | 1.5 | 0.7 | 1.7 | 0.8 | 1.7 | 0.7 | N | 1.2 | 5.6 | 0.6 | 8.8 | 27.6 |
| cZROG | N | N | N | N | N | N | 15.6 | 33.6 | 251 | 79.8 | 163 | 460 |
| Z7G | N | N | N | N | N | N | 2.4 | 2.4 | 18.0 | 5.2 | 5.4 | 45.8 |
| cZ7G | N | N | N | N | N | N | 6.4 | 6.6 | 5.6 | 8.0 | 2.8 | 4.6 |
| Z9G | N | N | N | N | N | N | 0.6 | N | 6.4 | 1.8 | 2.2 | 29.2 |
| cZ9G | N | N | N | N | N | N | 2.8 | 5.0 | 27.2 | 23.4 | 27.2 | 32.8 |
| DHZ | N | N | N | N | N | N | N | 0.6 | N | N | N | N |
| DHZR | N | N | N | N | N | N | N | N | N | N | N | N |
| DHZRP | N | N | N | N | N | N | N | N | N | 2.2 | 1.2 | N |
| DHZ7G | N | N | N | N | N | N | 1.2 | 1.4 | N | 2.0 | 0.4 | N |
| DHZ9G | N | N | N | N | N | N | N | 0.2 | N | 0.4 | 0.4 | N |
| DHZROG | N | N | 3 | 2.9 | N | N | 1.6 | 2.4 | N | 3.8 | 11.8 | 186 |

TABLE 1B

| | NONE | | | MEDIUM | | | STRONG | | |
|---|---|---|---|---|---|---|---|---|---|
| PHENOTYPE: | SR1 | N1 | N2 | M1 | M2 | M3 | ST1 | ST2 | ST3 |
| 2iP | N | N | 0.6 | N | 0.8 | N | N | 1.0 | 3.3 |
| 2iPR | N | N | N | 0.4 | 0.2 | N | 0.7 | 2.2 | 1.9 |
| 2iPRP | 0.6 | 1.0 | 2.7 | 2.8 | 4.3 | 3.4 | 6.6 | 11.5 | 11.8 |
| 2iP7G | 3.4 | 1.6 | 85.1 | 90.5 | 94.6 | 226 | 484 | 488 | 772 |
| 2iP9G | N | N | N | N | N | 0.2 | N | N | N |
| Z | N | N | N | N | 0.4 | N | N | N | N |
| cZ | N | N | 0.7 | N | N | N | 0.2 | N | N |
| ZRP | N | 0.3 | N | N | N | N | N | 0.1 | N |
| cZRP | N | N | N | 12.7 | N | N | 10.3 | 2.4 | N |
| ZOG | N | 0.3 | 0.2 | 0.2 | N | 1.3 | 0.7 | N | 2.4 |
| cZROG | 0.7 | 0.3 | 2.3 | 0.8 | N | N | N | 0.4 | N |
| Z7G | 1.4 | 1.2 | 1.2 | 1.2 | 0.8 | 1.3 | 4.5 | 1.4 | N |
| cZ7G | 6.1 | 2.0 | 8.7 | 10.2 | 13.2 | 7.2 | 6.7 | 8.5 | 11.5 |
| Z9G | N | 0.9 | N | N | N | N | N | 0.6 | N |
| DHZR | 0.4 | N | N | N | N | 0.3 | N | 0.6 | N |
| DHZRP | 0.7 | N | 0.3 | 0.5 | 0.8 | 0.9 | 70.7 | 0.5 | 4.6 |
| DHZ7G | 4.3 | 1.5 | 16.8 | 7.9 | 15.4 | 7.9 | 13.2 | 13.3 | 16.9 |
| DHZ9G | N | 0.5 | 0.6 | N | N | 0.3 | N | N | N |
| DHZROG | N | 0.3 | 1.2 | 0.4 | 0.5 | 0.2 | 0.8 | 0.4 | 0.2 |

### References

Binding H and Krumbiegel-Schroeren G (1984). Isolation and culture of protoplasts: Petunia. Cell Culture and Somatic Cell Genetics of Plants. V.1 340-349.

Fritze K and Walden R (1995). Gene activation by T-DNA Tagging in Methods in Molecular Biology V.44: Agrobacterium protocols Edited by: Kevan M. A.

Kakimoto T (1996). CKI1, a Histidine Kinase Homolog implicated in Cytokinin Signal Transduction. Science, Vol 274, 982-985.

Koes R E, Spelt C E, Mol J N M and Gerats A G M (1987). The chalcone synthase multigene family of Petunia hybrida (V30): sequence homology, chromosomal localization and evolutionary aspects. Plant Molecular Biology 10, 159-169.

Logemann J, Schell J and Willmitzer L, (1987). Improved method for the isolation of RNA from plant tissue. Analytical Biochemistry 163, 16-20.

Muller J F, Missionier C and Caboche M (1983). Low density growth of cells derived from Nicotiana and Petunia protoplasts; influence of the source of protoplasts and comparison of the growth-promoting activity of various auxins. Phisiol. Plant,57 35-41.

Van Blokland R, Ross S, Corrado G, Scollan S and Meyer P (1998). Development abnormalities associated with deoxyadenosine methylation in transgenic tobacco. The Plant Journal 15 (4), 543-551.

1. A nucleic acid molecule comprising the sequence set forth in SEQ ID NO:1 or a part thereof, or homologue thereof, the sequence encoding a cytokinin biosynthesis enzyme and which predominantly encodes 2iP derivatives.

2. A nucleic acid molecule according to claim 1 which hybridises under high stringency to SEQ ID NO:1.

3. A nucleic acid according to either claim or 2 which is isolated.

4. A nucleic acid according to any preceding claim which is of plant origin.

5. A nucleic acid encoding a cytokinin biosynthesis enzyme and which predominantly encodes 2iP derivatives, the nucleic acid may be selected from the group consisting of:
(a) DNA having the nucleotide sequence given herein as SEQ ID NO:1 (which encodes the protein having the amino acid sequence given herein as SEQ ID NO:2), and which encode a cytokinin biosynthesis enzyme, predominantly 2iP derivatives;
(b) nucleic acids which hybridise to DNA of (a) above (e.g., under stringent conditions) and which encode a cytokinin biosynthesis enzyme, predominantly 2iP derivatives; and
(c) nucleic acids which differ from the DNA of (a) or (b) above due to the degeneracy of the genetic code, and which encode a cytokinin biosynthesis predominantly in the form of 2iP derivatives enzyme encoded by a DNA of (a) or (b) above.

6. A nucleic acid molecule comprising the sequence set forth in SEQ ID NO:7 or a part or variant thereof or a homologue thereof, which acts as a promoter for a nucleic acid encoding a cytokinin biosynthesis enzyme and which predominantly encodes 2iP derivatives.

7. A vector which has inserted therein a nucleic acid molecule according to any preceding claim.

8. A vector according to claim 7 which is bacterial or viral in origin.

9. A vector according to either claim 7 or 8 which is an Agrobacterium transformation vector.

10. A vector according to any one of claims 7-9 further including an enhancer element to stimulate transcriptional activity.

11. A vector according to claim 10 wherein the enhancer is capable of stimulating transcriptional activity by at least a 10 fold over basal level transcriptional activity.

12. A vector according to claim 10 wherein the enhancer stimulates transcription 10-100 fold over basal level transcriptional activity.

13. A vector according to claim 10 wherein the enancer stimulates transcription greater than 100 fold over basal level transcriptional activity.

14. A vector according to any one of claims 10-13 wherein the enhancer element is a 20 bp transcriptional sequence position −90 to −427 from the RB (right border sequence) of a 35S RNA promoter of cauliflower mosaic virus.

15. A vector according to any one of claims 7-14 further including a promoter.

16. A vector according to claim 15 wherein the promoter is cell/tissue specific inducible or constitutive.

17. A vector according to either claim 15 or 16 wherein the promoter is a 35S RNA promoter of cauliflower mosaic virus.

18. A vector according to any one of claims 15-17 wherein the nucleic acid molecule of SEQ ID NO:1 is fused to a reporter gene domain of the promoter.

19. A polypeptide or protein of SEQ ID NO:2 or functionally equivalent part or analogue or derivative thereof.

20. Use of a nucleic acid according to any one of claims 1-6 and/or polypeptide or protein encoded thereby in regulation of any one or more of the following processes: plant growth, shoot development, cell division, cell cycles, cell activity, stimulation of nutrient mobilisation, delay of senescence both in vivo and post harvesting, lateral branching, improvement of shelf-life, morphogenesis, increased insect/pathogen tolerance and/or light initiated maturation of chloroplasts.

21. Use of a nucleic acid according to any one of claims 1-6 and/or a polypeptide or protein encoded thereby or vector according to any one of claims 7-18 in the activation of a target gene or genes.

22. Use of a nucleic acid according to any one of claim 1-6 and/or a polypeptide or protein encoded thereby or vector according to any one of claims 7-18 in regulating flood tolerance in plants.

23. A method of regulating any one or more of the following processes, plant growth, shoot development, cell division, cell cycles, cell activity, stimulation of nutrient mobilisation, delay of senescence both in vivo and post harvesting, lateral branching, morphogenesis, increased pathogen tolerance, improved shelf-life, light initiated maturation of chloroplasts, activating a target gene or genes or regulating flood tolerance comprising genetically engineering a plant cell or tissue or plant or seed so as to incorporate into its genome or cause to express above a basal level a nucleic acid according to any one of claims 1-6.

24. Use of a vector according to any one of claims 7-18 in identifying a marker gene.

25. A plant cell and/or a plant tissue and/or plant and/or plant seed containing a transcriptionally activated/activatable form of a nucleic acid molecule according to any one of claims 1-6, a vector according to any one of claims 7-18 or a fragment of such a vector which comprises a residue of a nucleic acid molecule of any one of claims 1-6.

26. A plant cell and/or plant tissue and/or plant and/or plant seed having integrated or inserted into its genome at least one more copy of a sequence encoding a nucleic acid molecule according to any one of claims 1-6 than its naturally occurring counterpart.

27. A plant cell and/or plant tissue and/or plant and/or plant seed according to claim 26 comprising multiple copies of the encoding sequence.

28. A plant cell and/or plant tissue and/or plant and/or plant seed according to either claim 24 or 25 comprising 1, 2, 3, 4, 5, 6, 7, 8, 9 or 10 or more copies of the encoding sequence

29. A plant material selected from a plant cell and/or plant tissue and/or plant and/or plant seed whose genome contains recombinant DNA comprising the sequence of a nucleic acid of any of claims 1 to 6.

30. A plant material of claim 29 wherein the recombinant DNA is flanked by foreign nucleic acid sequences.

31. A plant material of claim 29 or claim 30 wherein the recombinant DNA is operatively associated with an enhancer.

32. A recombinant transgene comprising a plant nucleic acid sequence having inserted therein a nucleic acid of any of claims 1 to 6, and optionally including the specific feature of claim 30 or claim 31.

33. A plant generated from a plant cell and/or plant tissue and/or plant seed which contains at least one additional copy of a sequence encoding a nucleic acid molecule according to any one of claims 1-6 as compared to the wild type.

34. A plant according to any one of claims 29 to 31 wherein the plant is a cereal crop or grass.

35. Use of a vector according to any one of claims 7-18 or a recombinant transgene of claim 32 or use of enhanced expression of a nucleic acid according to any one or more of claims 1-6 in the production of plants with any one or more of the following characteristics:
(i) reduced apical dominance;
(ii) hormone-independent regeneration of shoot from calli and leaves;
(iii) reduced height (internode length);
(iv) increased leaf/stem ratios
(v) delayed flowering
(vi) delayed senescence
(vii) increased insect tolerance
(viii) increased flooding tolerance
(ix) increased life span of the plant
(x) increased vase and shelf life of plant parts and;
(xi) increased pathogen tolerance.

36. A method of producing a genetically modified plant with any one or more of the characteristics recited in claim 35 comprising enhancing expression of a nucleic acid according to any one of claims 1-5 or a promoter according to claim 6 or a vector according to any one of claims 7-18, or causing it to have a recombinant transgene of claim 32 in its genome.

37. Use of the nucleic acid according to any one of claims 1 to 5 or the promoter of claim 6 as a probe for selection of plants that exhibit enhanced levels of CK biosynthesis enzymes predominantly in the form of 2iP derivatives.

38. Use according to claim 37 for selecting naturally occurring plant varieties that have high endogenous levels of CK biosynthesis enzymes predominantly in the form of 2iP derivatives.

39. Use as a cosmetic of nucleic acid according to any one of claims 1-6 and/or plant, plant cell and/or a plant tissue and/or plant and/or plant seed and/or plant extract containing a transcriptionally activated/activatable form of the nucleic acid molecule or whose genome contains recombinant DNA comprising the sequence of a the nucleic acid.

40. The use in the preparation of a cosmetic of a nucleic acid according to any one of claims 1-6 and/or plant, plant cell and/or a plant tissue and/or plant and/or plant seed and/or plant extract containing a transcriptionally activated/activatable form of the nucleic acid molecule or whose genome contains recombinant DNA comprising the sequence of a the nucleic acid.

41. A cosmetic preparation comprising a nucleic acid according to any one of claims 1-6 and/or plant, plant cell and/or a plant tissue and/or plant and/or plant seed and/or plant extract containing a transcriptionally activated/activatable form of the nucleic acid molecule or whose genome contains recombinant DNA comprising the sequence of a the nucleic acid and further including a suitable carrier, excipient, diluent or base or foundation product.

42. A cosmetic preparation according to claim 41 further comprising molecules selected from the group consisting of elastin, elastin fragments, elastin-glycolie acid, collagen, collagen fragments, yeast extacts, skin respiratory factor, glucosamine, glucosamine sulfate, hyaluronic acid, hyaluronate, chondroitin sulfate, cholic acid, deoxycholic acid, ginseng extract, aloe vera powder, aloe vera oil, RNA and DNA fragments, ascorbyl palmitate, ascorbic acid, retinal palmitate, 7-dehydroxy cholesterol, vitamine E tocopherol, vitamin E lineolate, panthenyl ethyl ester, glycerol ceramides, glycogen, DL-pyroglutamic acid, urea, sodium lactate, lactate, glycerin, sorbitol, oils of borage, evening primrose, black currant, almond and canola, vanishing cream, cholesterol, flavenoids, witch hazel, chamomile, parsley, hibiscus, capric and caprylic triglycerides, amino acids, allantoin, sodium, calcium, potassium, phosphate, chloride, sodium lactate, alpha hydroxy acids, cocoa butter, coconut oil, jojoba oil, safflower oil, wheat germ oil, sesame oil, selachyl alcohol, shark oil, cerebrosides, proanthocyanidin, farnestol, candelellila, carnuba wax, vitamin E, nicotinate, manganese ascorbate, zinc, oleyl alcohol, polysorbate 80, spermaceti, glycerol monostearate, beeswax, silicone oil, paraffin wax, ozokerit E, and PEG 75 lanolin.

43. A cosmetic preparation according to either claim 41 or 42 wherein the preparation is in the form of a lotion, salve, cream, liposome, spray, micelle or gel.

44. A method of improving skin/hair/fingernail/toenail condition or moisturising skin hair/fingernails/toenails comprising applying topically an effective amount of a preparation comprising a nucleic acid according to any one of claims 1-6 and/or plant, plant cell and/or a plant tissue and/or plant and/or plant seed and/or plant extract containing a transcriptionally activated/activatable form of the nucleic acid molecule or whose genome contains recombinant DNA comprising the sequence of a the nucleic acid.

45. A method of reducing wrinkles in keratinous structures and/or other ageing effects of the skin, moisturising or improving the condition of skin/hair/fingernail/toenail comprising applying topically an effective amount of a preparation comprising a nucleic acid according to any one of claims 1-6 and/or plant, plant cell and/or a plant tissue and/or plant and/or plant seed and/or plant extract containing a transcriptionally activated/activatable form of the nucleic acid molecule or whose genome contains recombinant DNA comprising the sequence of a the nucleic acid.


SEQUENCE LISTING (7 sequences)

SEQ ID NO: 1 - DNA, 1053 bp, Petunia hybrida
  CDS (1)..(1050)
atgttaattgtagtacatattattagcatcacacgcatcatattcatcaccttaacccataatcatctccatttccttatgtttagatcattatcatacaatcacaagcacctcaaattccttacaaacccgaccacacgggtactccgaagaaacatgtcgtcatccactgtagtaacaatacccggccccacacaaaaaaacaaaaacaaaatcatagtaataatgggtgcaacaggttcaggaaaatcaaaactctcaatagacctcgtcacacgtcactatcctttttccgaaatcattaactccgacaaaatccaaattaccaaaggtttaaacataaccacaaacaaaatcactgtacccgaccgacgtggcgtagttcatcatttactcggcgagattgaccccgactttaacttttctccttctcatttccggtcaattgctggtcaacgcattaactccattattaatcgccataaactcccattcctcgttggtgggtccaactcatatatctacgctttattaacaaaccggttcgacccggattttaaccctgattcaaacccggttcattttatatccaacgagttacgctacaactgttgttttatttgggtcgatgtattaaacccggttttgaatgagtatttggataaacgggtcgatgagatgatgaactcgggtatgtatgaagaactggaacagttttttaaagaaaacaggttttcggatccgggtttggaaccgggtcgggccaccgggttgaggaaagcgataggggtaccggaaatggagaggtattttaagaagagctgtacgtatgaggaagcagtgagggaaataaaagaaaacacgtggcggttagcgaagaagcagatgtggaagatccaacggttgagagaagcagggtgggacctacaaagagtagatgccacggaggcatttgtggaggcgatgagtaataagaaggaaaagggaattatttgggaaaaacaagtagtggaaccaagtgtcaagattgtgaaccgttttttgttggactga

SEQ ID NO: 2 - protein, 350 aa, Petunia hybrida
MLIVVHIISITRIIFITLTHNHLHFLMFRSLSYNHKHLKFLTNPTTRVLRRNMSSSTVVTIPGPTQKNKNKIIVIMGATGSGKSKLSIDLVTRHYPFSEIINSDKIQITKGLNITTNKITVPDRRGVVHHLLGEIDPDFNFSPSHFRSIAGQRINSIINRHKLPFLVGGSNSYIYALLTNRFDPDFNPDSNPVHFISNELRYNCCFIWVDVLNPVLNEYLDKRVDEMMNSGMYEELEQFFKENRFSDPGLEPGRATGLRKAIGVPEMERYFKKSCTYEEAVREIKENTWRLAKKQMWKIQRLREAGWDLQRVDATEAFVEAMSNKKEKGIIWEKQVVEPSVKIVNRFLLD

SEQ ID NO: 3 - DNA, 24 bp, Artificial sequence
  Oligonucleotide primer
acatgtcgtcatccactgtagtaa

SEQ ID NO: 4 - DNA, 25 bp, Artificial sequence
  Oligonucleotide primer
aggttttcggatccgggtttggaac

SEQ ID NO: 5 - DNA, 24 bp, Artificial sequence
  Oligonucleotide primer
gtattatacaatccaaagattgag

SEQ ID NO: 6 - DNA, 25 bp, Artificial sequence
  Oligonucleotide primer
caccaaaatgaactacagtgggata

SEQ ID NO: 7 - DNA, 252 bp, Petunia hybrida
  promoter (1)..(252)
atctagatccgaaactatatatacacacacaaatgaaaaagctccatcttcatcctcatcaaaacaacacaactttctctgatcataacagaaatactagctagtacaacatatcccattcccacccaactccttcatatagtgctagctcaggtgctgctattttccagactagtagtactattctgcacaaacaagtaaacaaccccacccccacccattttcccctctaccatttgtcatgttttccgt